(19) United States
(12) Patent Application Publication
(10) Pub. No.: US 2024/0164827 A1
(43) Pub. Date: May 23, 2024

(54) ELECTROSURGICAL FORCEPS WITH DISPLACEABLE HEAT SINK FOR THERMAL CUTTING

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Jordan R. Golomb, Maple Grove, MN (US)

(21) Appl. No.: 18/516,617

(22) Filed: Nov. 21, 2023

Related U.S. Application Data
(60) Provisional application No. 63/384,660, filed on Nov. 22, 2022.

(51) Int. Cl. A61B 18/08

(52) U.S. Cl. CPC A61B 18/085; A61B 2018/00601

(57) ABSTRACT

An electrosurgical medical device can include a first jaw, a second jaw, a heating element, and a heat sink. The heating element can be heated for cutting or sealing tissue. The heat sink and the heating element can move relative to each other. Such relative movement can be between a first state that dissipates heat from the heating element and a second state that allows the heating element to heat. Such relative movement can be actuated during closing of the jaws. For example, a displacement actuator on the first jaw can push the heat sink away from the heating element when the jaws are being closed, thereby moving the heating element and the heat sink from a first (heat sinking) state to a second (non-heat sinking) state. Heat in the heating element can dissipate through the heat sink in the first state for rapid cooling of the heating element.

### PRIORITY CLAIM

This application claims the benefit of priority to U.S. Provisional Patent Application Ser. No. 63/384,660, filed Nov. 22, 2022, the contents of which are hereby incorporated by reference.

### TECHNICAL FIELD

This document pertains generally, but not by way of limitation, to medical devices, and more particularly to an electrosurgical device that can include or combine thermal, electrical, and/or mechanical modalities for producing an effect on an anatomical feature.

### BACKGROUND

Bipolar electrosurgical devices can be used for cutting a patient's tissue during surgical procedures. Bipolar electrosurgical devices can include an end effector, such as forceps that can include a jaw assembly with a mechanical clamping action to grip, capture, grasp, manipulate, pull, constrict, cut, and/or dissect an anatomical feature such as a vessel or tissue. They can also include electrosurgical capabilities so the anatomical feature can be sealed, cut, or coagulated.

In addition to sealing, cutting or coagulating, some forceps can also be used to dissect an anatomical feature, such as via one or more blunt dissection techniques. One blunt dissection technique is sweep dissection. In sweep dissection, the jaw assembly, or a portion of the jaw assembly, such as an edge, is moved or “swept” across the anatomical feature thereby dissecting or separating the anatomical feature from another anatomical feature.

### SUMMARY/OVERVIEW

Forceps that use an electrosurgical therapy current to cut tissue can generate and retain heat, such as in or near electrodes within the jaws of the forceps. This can cause unintended thermal damage to nearby tissue during sweep dissection or other maneuvering of the jaw assembly. Improvements in such devices are desired.

This document pertains to an electrosurgical device such as for cutting or sealing tissue of a patient. The electrosurgical device can include an end effector such as bipolar forceps. The forceps can include a jaw assembly that can move between an open position and a closed position. For example, the jaw assembly can include a first jaw and a second jaw. At least one of the first jaw and the second jaw can include a heating element and a heat sink. The heating element can heat to a temperature sufficient for cutting or sealing the tissue of the patient.

In an open position, the jaw assembly can be maneuvered such that a tissue of a patient is positioned between the first jaw and the second jaw. In the open position, the heat sink, the heating element, or both can move relative to each other, such as between a first state and a second state. In a first state, a heat-sinking position can be provided, such as for cooling the heating element. In the first state, a portion of the heat in the heating element can dissipate through the heat sink. When the jaws are maneuvered into a closed position, such as to grasp the tissue, the heating element and the heat sink can be displaced relative to each other into a second state, in which the heating element and the heat sink are at least partially thermally decoupled, such as to allow the heating element to deliver heat to the tissue without sinking any, or as much, heat through the heat sink. In the second state, the heating element can cut or seal the tissue.

### BRIEF DESCRIPTION OF THE DRAWINGS

FIG. 1A illustrates a side view of a forceps showing a jaw assembly in an open position.

FIG. 1B illustrates a side view of the forceps of FIG. 1A showing the jaw assembly in a closed position.

FIG. 2A is a side view of a jaw assembly in an open position.

FIG. 2B is a side view of the jaw assembly of FIG. 2A in a closed position.

FIG. 3 is a cross section of the jaw assembly of FIG. 2A, taken along line 2 in FIG. 2A.

FIG. 4A is a side view of an electrosurgical device in a first state with a heat sink configured in a position adjacent to the heating element.

FIG. 4B is a side view of the electrosurgical device of FIG. 4A in a second state with a heat sink configured to retract downward and away from a heating element.

FIG. 5 is a side view of the forceps of FIG. 1A in an open position with a patient's tissue positioned across the heating element.

FIG. 6 is a flow chart of a method of treating tissue with an electrosurgical device.

FIG. 7 is a flowchart indicating a reprocessing method for the electrosurgical device of FIGS. 2A, 2B, 3, 4A, and 4B.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

This document describes an electrosurgical device that can include a handpiece that operates a forceps that includes an end effector, such as a jaw assembly including a heating element, such as for cutting or sealing of tissue in surgical procedures, and a heat sink that is displaceable relative to the heating element, such as to at least partially dissipate heat from the heating element when not displaced and to be spaced apart from the heating element when displaced. When not so displaced, the displaceable heat sink can help rapidly reduce temperature of the heating element, such as between cutting or sealing actions. This can help reduce the risk or extent of unintended damage to a patient's nearby tissues, such as when the electrosurgical device is moved about within a surgical field. The end effector of the device can include a jaw assembly comprising a first jaw and a second jaw. A heating element can be coupled to or disposed within the second jaw. A displaceable heat sink can be coupled to or disposed within the second jaw, which can be placed into contact with the heating element when heat sinking is desired and displaced to be spaced apart from the heating element when the heating element is being used to treat target tissue such as being grasped within the jaw for tissue cutting or sealing.

A surgeon may control actuation of the end effector of the forceps to actuate one or more functions of the end effector. Actuation of the end effector can be facilitated by one or more actuation systems of a handpiece that can retract, extend, or rotate one or more shafts to control the actions of the end effector. The jaw assembly can be controlled by the handpiece including an actuation system to be one or more of: rotatable, openable, closeable, extendable, and capable of supplying electromagnetic energy. Other handpieces can be connected to and can control the end effectors described herein. This disclosure includes examples of handpieces including one or more actuation systems and examples where the disclosed actuation systems and end effectors can be used together in a medical device.

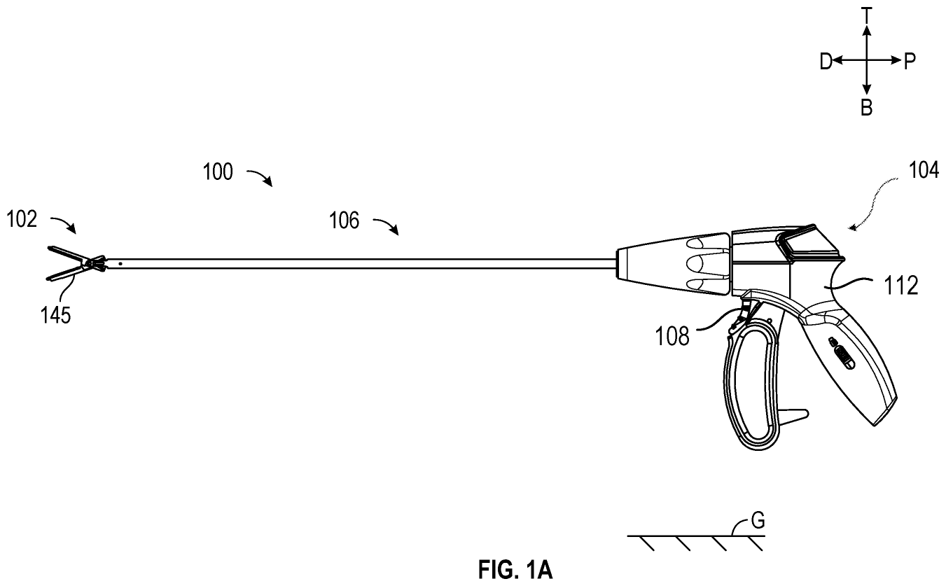

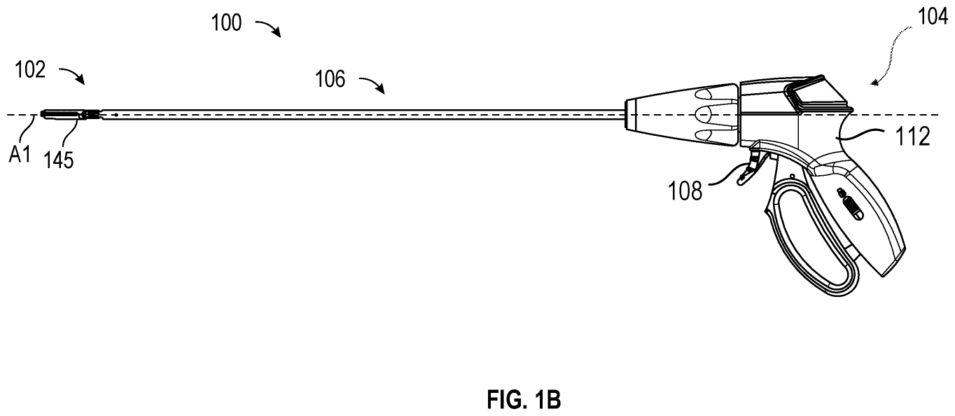

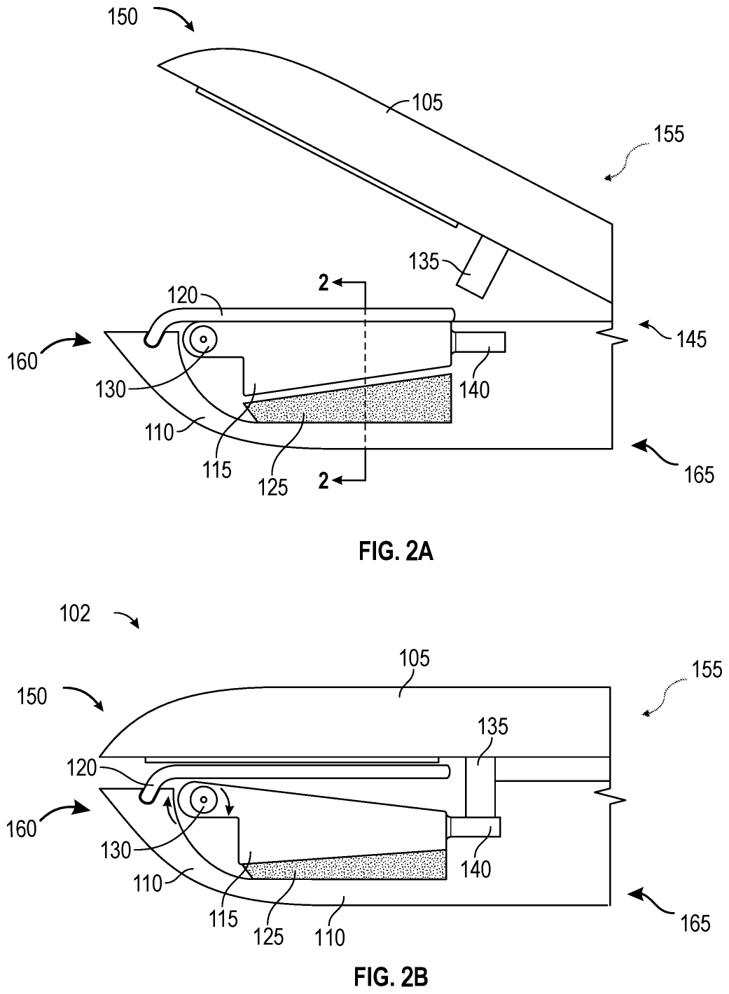

FIG. 1A illustrates a side view of an electrosurgical device 100 with a jaw assembly 145 in an open position. FIG. 1B illustrates a side view of the electrosurgical device 100 with the jaw assembly 145 in a closed position. FIG. 2 illustrates an exploded view of some components of the jaw assembly 145 of the electrosurgical device 100 of FIG. 1A. FIGS. 1A and 1B are described together. Directional descriptors such as proximal and distal are used within their ordinary meaning in the art. The proximal direction P and distal direction D are indicated on the axes provided in FIG. 1A, which are defined when the electrosurgical device 100 is held level with respect to a ground G in an upright orientation as shown in FIG. 1A. Opposite to the lateral directions L and L′, is the medial direction, in other words, the medial direction is towards the centerline, or a longitudinal axis A1 of the electrosurgical device 100 (FIG. 1B).

The illustrative electrosurgical device 100 can include a handpiece 104 at a proximal end, and an end effector 102 at a distal end. An intermediate portion 106 can extend between the handpiece 104 and the end effector 102 to operably couple the handpiece 104 to the end effector 102. Various movements of the end effector 102 can be controlled by one or more actuation systems of the handpiece 104. In the illustrative example, the handpiece 104 can include a housing 112 and a trigger 108.

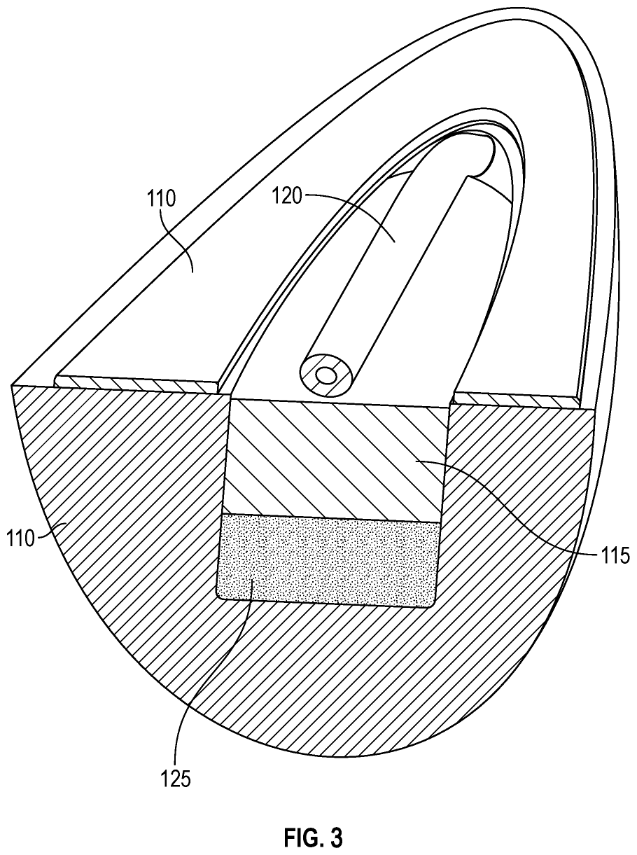

In the illustrative example, the end effector 102 can include a jaw assembly 145 that is capable of opening and closing. The end effector 102 can be rotated along a longitudinal axis A1 (FIG. 1B) of the electrosurgical device 100. In this example, the end effector 102, or a portion of the end effector 102 can be one or more of: opened, closed, rotated, extended, retracted, and electromagnetically energized (e.g., electrically energized). In some examples, the energy can be radio-frequency energy. The end effector 102 can include a heating element 120 that can include one or more resistive electrodes configured to seal or cut the target tissue of the patient (FIGS. 2A, 2B, and 3). All actuation system functions, and all end effector actions are not required in all examples. The functions described herein can be provided in any combination.

The handpiece 104 can enable a user to at least partially or fully open or close the jaw assembly 145. For example, the jaw assembly 145 can be in a closed position when the trigger 108 is in default position distal to the handle 104, as shown in FIG. 1B. The jaw assembly 145 can be opened by displacing the trigger 108 proximally, as shown in FIG. 1A. The handpiece 104 can also be alternatively configured such that the jaw assembly 145 can be in an open position when the trigger 108 is in the default position distal to the handle 104. The jaw assembly 145 can be closed by displacing the trigger 108 proximally.

FIG. 2A shows a side view of a jaw assembly 145 in an open position. The electrosurgical device 100 can include a bipolar forceps including a jaw assembly 145 that can move between a closed position and an open position. The jaw assembly 145 comprises a first jaw 105 and a second jaw 110. The first jaw 105 can have a first distal end 150 and a first proximal end 155. The second jaw 110 can have a second distal end 160 and a second proximal end 165. The first proximal end 155 can be movably coupled to the second proximal end 165 to provide an end effector forceps including the first jaw 105 and second jaw 110.

The first jaw 105 and the second jaw 110 can move between an open position (shown in FIG. 2A) and a closed position (shown in FIG. 2B). The first jaw 105 and the second jaw 110 can be in a partially open position as the first distal end 150 of the first jaw 105 moves away from the second distal end 160 of the second jaw 110. The first jaw 105 and the second jaw 110 can stop in any partially open position as the user manipulates the trigger 108. For example, the user may manipulate the trigger 108 to stop the first jaw 105 and the second jaw 110 in or between a 10%, 20%, 30%, 40%, 50%, 60%, 70%, 80%, 90%, or less than 100% partially open position to maneuver the electrosurgical device 100 within a body cavity of the patient and position the electrosurgical device 100 around the tissue to be cut or sealed. As described herein, an open position of the first jaw 105 and the second jaw 110 can be any partially open position or 100% fully open position wherein the first distal end 150 of the first jaw 105 moves (or has moved and is paused) away from the second distal end 160 of the second jaw 110 and the heating element 120 is cooling through heat dissipation by the heat sink 115.

The first jaw 105 and the second jaw 110 can be in a partially closed position as the first distal end 150 of the first jaw 105 moves toward the second distal end 160 of the second jaw 110. The first jaw 105 and the second jaw 110 can stop in any partially closed position as the user manipulates the trigger 108. For example, the user can manipulate the trigger 108 to stop the first jaw 105 and the second jaw 110 in or between a 10%, 20%, 30%, 40%, 50%, 60%, 70%, 80%, 90%, or less than 100% partially closed position to grasp the patient's tissue and effect the desired cutting or sealing of the tissue. As described herein, a closed position of the first jaw 105 and the second jaw 110 can be any partially closed position or 100% fully closed position wherein the first distal end 150 of the first jaw 105 moves toward the second distal end 160 of the second jaw 110 and the heating element 120 is heating and is at least partially decoupled from the heat sink 115.

The first jaw 105, the second jaw 110, the heat sink, or a combination thereof can include one or more coatings to aid in ease of use during surgical procedures. For example, the first jaw 105 and the second jaw 110, including the heating element 120 or the heat sink 115 described herein, can be coated in a non-stick coating to reduce or prevent the jaw assembly 145 from sticking to the patient's tissues. In an example, the coating can comprise a moisture wicking material to help transport moisture away from the target tissue site being treated, if desired. The coating can reduce or prevent a phase change of the patient's body fluids to steam as the heat sink 115 and the resilient bias member 125 (described below) dissipate a portion of heat away from the heating element 120. Examples of suitable materials include polytetrafluoroethylene (PTFE), hexamethyldisiloxane (HMDSO), ceramics, aluminum oxide and other materials that work well under high temperatures. Although such coatings can be thermally insulative, the inventors discovered that if provided sufficiently thin, the coating can remain thermally conductive enough to transmit heat.

The heating element 120 can be coupled to or disposed within the second jaw 110. The heating element 120 can include one or more resistive electrodes configured to seal or cut the target tissue of the patient when the target tissue of the patient is placed between the first jaw 105 and the second jaw 110 in the closed position of FIG. 2B. The resistive electrode can be configured to receive electrical energy from a power source (such as a conventional electrosurgical generator) to deliver electromagnetic energy (e.g., radiofrequency, alternating current (AC)) to the tissue to seal or cut the patient's tissue. The energy may also be provided in a direct current (DC) form of energy.

The heating element 120 can be resistively heated, such as by being provided with electrical power, heating power, therapeutic power, one or more signals, or a combination thereof from any source to resistively heat the resistive electrode. The heating element can be heated to a range of approximately 200° C. to approximately 350° C. to provide resistive heat cutting capability. For example, in some cases, the heating element can be heated to a range of approximately 260° C. to approximately 300° C. In low-temperature electrocautery medical devices, the heating element can be heated to a temperature between approximately 40° C. to approximately 70° C.

The heat sink 115 can be disposed within the second jaw 110, such as adjacent to the heating element 120 as shown in FIG. 2A. FIG. 3 shows a cross section along line 2 shown in FIG. 2A. A heat sink is a type of heat exchanger, such as a passive heat exchanger that transfers heat away from another portion or component of a device to draw heat away and cool that portion or component of the device. Fourier's law of heat conduction shows that when there is a temperature gradient in a body, heat will be transferred from the higher-temperature region to the lower-temperature region.

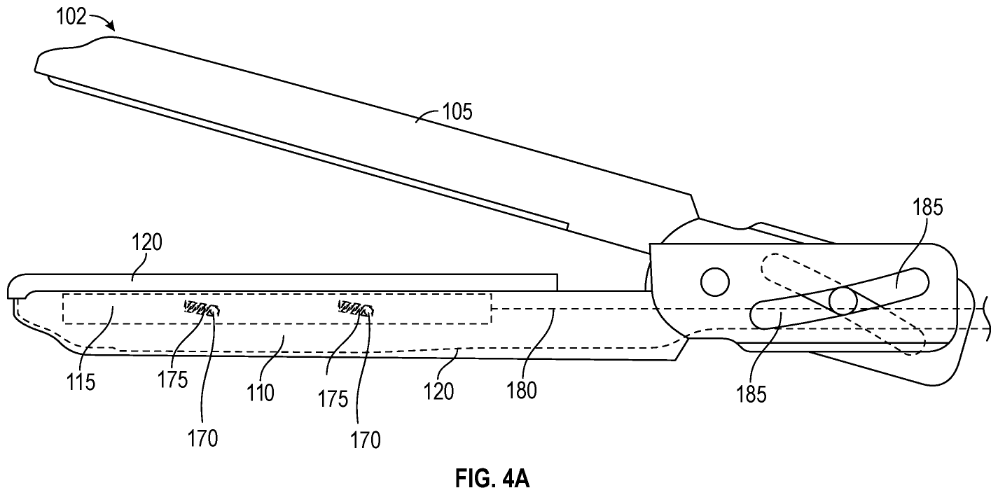

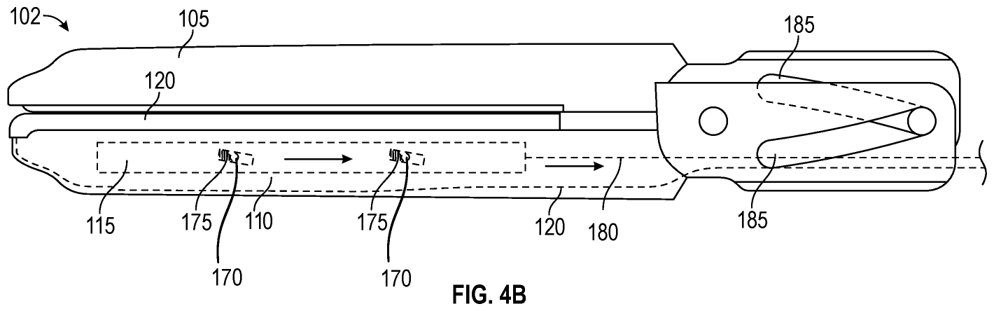

As shown in the example of FIGS. 2A, 2B, 3, 4A, and 4B, the second jaw 110 of the electrosurgical device 100 can include the heat sink 115, which can be positioned adjacent to the heating element 120. The heat sink 115 can be disposed within the second jaw 110 and can move relative to the heating element 120 to selectively dissipate a portion of heat away from the heating element 120. In a first state, the heating element 120 can be in a contacting or another at least partially thermally coupled state with the heat sink 115 as shown in FIG. 2A and FIG. 4A. In some cases, the at least partially thermally coupled state can include close proximity between the heat sink 115 and the heating element 120. In some cases, the at least partially thermally coupled state can include direct contact between the heat sink 115 and the heating element 120. In a second state, as shown in FIG. 2B and FIG. 4B, the heat sink 115 can move away from the heating element 120 to a less thermally coupled state relative to the at least partially thermally coupled state of the first state. The second state can include, but is not limited to, a thermally de-coupled state that allows the heating element 120 to heat to a temperature sufficient for cutting, sealing or coagulating of the patient's tissue.

In the first state, the heat sink 115 can be in an at least partially thermally coupled state with the heating element 120 to promote efficient dissipation of a portion of the heat away from the heating element 120, such as for rapid cooling to a temperature that cannot effect cutting, sealing, or damage to a patient's tissue. For example, in some cases, placing the heat sink 115 in the first state with respect to the heating element 120 can reduce the temperature of the heating element 120 to approximately the local body ambient temperature within approximately 0.25 seconds to approximately 3 seconds, such as one second. In some examples, depending on the size and material properties of the heating element 120 and the heat sink 115, the time range can be between 0.1 and 1.5 seconds to sufficiently cut the tissue while maximizing the speed at which the heating element 120 cools.

The temperature of the heating element 120 upon heat dissipation in the first state can depend on the local ambient temperature and can be impacted by the number of times the heating element 120 has been heated in the second state. If the heating element 120 has been heated extensively and repeatedly, the temperature of the heating element 120 may be elevated relative to ambient temperature when cooled in the first state. With such cooling, however, the elevated temperature cannot affect cutting, sealing, or damage to a patient's tissue. For example, the elevated temperature may be below approximately 42° C., such as between approximately 37° C. and approximately 42° C.

Rapid cooling of the heating element 120 can help prevent unintended heat effects to surrounding tissues that may be inadvertently touched with heating element 120. Rapid cooling of the heating element 120 can also help inhibit or prevent burns to tissues from a phase change of the patient's body fluids into steam when the body fluids come into contact with the heating element 120. Rapid cooling can also help allow a rapid transition of use of the electrosurgical device from cutting to sealing tissue, which can use a lower temperature than cutting.

In some examples of this disclosure, the heat sink 115 can include an intentional heat sink arranged to intentionally draw the heat away from a heating element. In some examples, the heat sink's 115 primary purpose or only purpose in the electrosurgical device 100 can be to transfer heat away from the heat sink 115.

The heat sink 115 and the heating element 120 can be arranged and move with respect to one another by various mechanisms. The examples provided herein are provided merely for illustration, and other mechanisms to displace the heat sink and the heating element relative to one another can be employed within the scope of this disclosure.

In the first state, the heat sink 115 can actively or passively cool the heating element 120. A passive heat sink 115 can comprise any suitable thermally conductive material for dissipating a portion of the heat in the heating element 120. In some cases, the passive heat sink 115 can have a thermal conductivity that is greater than a thermal conductivity of the heating element 120. For example, the passive heat sink 115 can comprise one or more of aluminum, copper, or an engineered graphite foam or like material. In some cases, the passive heat sink 115 can be less thermally conductive, or a similarly thermally conductive, to the heating element 120. The lower temperature of the passive heat sink 115 as compared to the heating element 120 can draw away thermal energy from the heating element 120 via natural thermal conduction across a thermal gradient from a high temperature to a low temperature.

The heat sink 115, the heating element 120, or both can move relative to each other between the a first state and the second state. When the first jaw 105 and the second jaw 110 are in the closed position or moved toward the closed position, the first jaw can actuate displacement of the heat sink 115 into the second state, as shown in FIG. 2B, and the heating element 120 can then be energized to deliver heat to tissue captured between the first jaw 105 and the second jaw 110. When the first jaw 105 and the second jaw 110 are in the open position or moved toward the open position, the heating element 120, the heat sink 115, or both can be moved into the second state to dissipate heat away from the heating element 120.

The heat sink 115 can be pivotably (as shown in FIGS. 2A-B) attached to the end effector 102, such as to the second jaw 110, such as at an opposite end of the second jaw 110 from the displacement actuator 135. A pivot member 130 can be coupled to the heat sink 115 to provide a pivot point for the heat sink 115 to move toward the heating element 120 into the first state when the first jaw 105 and second jaw 110 are in the open position.

In another example, the heat sink 115 can be slidably attached to the second jaw 110 (as shown in FIGS. 4A-B). The heat sink 115 can be coupled to the second jaw 110 with any suitable fastener that provides the heat sink 115 with a sliding movement. For example, the fastener can be a pin 170 coupled to a compressible member 175, such as a spring or compressible material. The heat sink 115 can slide into a position adjacent to the heating element 120 for heat dissipation in the first state (FIG. 4A) as the first jaw 105 and second jaw 110 are opened through movement of a hinge 185, wherein the compressible member 175 is at least partially decompressed. The heat sink 115 can slide or otherwise retract downward and away from the heating element 120 in the second state, such as by retracting cord 180 to allow the heating element 120 to heat (FIG. 4B). In this retracted position, the compressible member 175 can be compressed until released to again slide into the position adjacent to the heating element 120 in the first state. The examples described herein are merely for illustration, any type of movement to displace the heat sink may be provided.

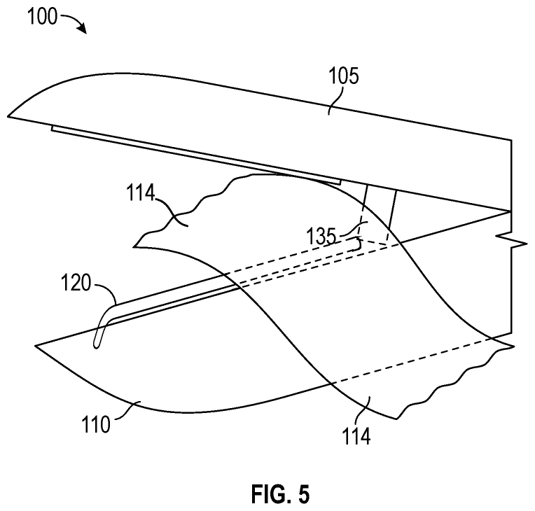

Referring again to FIGS. 2A-B, the first jaw 105 can include a displacement actuator 135, such as a pillar or other protrusion from a face of the first jaw 105 (e.g., finger, tab). The displacement actuator 135 can engage the heat sink 115 or a displacement member 140 that can be included in or connected to the heat sink 115, such as at an end of the heat sink 115 that is away from the pivot 130. This engagement can occur when the first jaw 105 and the second jaw 110 are moved toward the closed position, such as to pivot the heat sink 115 toward the second state. In some examples, as shown in FIG. 5, the displacement actuator 135 can also act as a tissue stop such as to help inhibit or prevent the patient's tissue 114 from advancing further proximally into the jaw assembly 145 when the displacement actuator 135 is located at the second proximal end 165 of the second jaw 110.

A resilient bias member can be provided to bias the heat sink 115 toward the heating element 120 in the first state when the first jaw 105 and the second jaw 120 are in the open position. For example, as shown in the illustrative example of FIGS. 2A and 2B, the resilient bias member 125 can be located adjacent to the heat sink 115 in the second jaw 110. When the first jaw 105 and the second jaw 120 are drawn towards one another during closing, the displacement actuator 135 can push the displacement member 140 in a direction away from the heating element 120, thereby causing the heat sink 115 to be displaced away (e.g., pivot) and be at least partially thermally de-coupled from the heating element 120 (e.g, mostly thermally de-coupled, substantially completely de-coupled, or completely thermally de-coupled from the heating element). When the heat sink 115 pivots away from the heating element 120, the heat sink 115 can push against the resilient bias member 125, such as to compress the resilient bias member 125 such as shown in FIG. 2B. With the heating element 120 and heat sink 115 at least partially thermally decoupled, the heating element 120 can then be energized to deliver heat to tissue captured between the first jaw 105 and the second jaw 110. See FIG. 5 showing tissue 114 disposed over the heating element 120 and between the first jaw 105 and the second jaw 110. When the first jaw 105 and the second jaw 110 are again in the open position, as shown in FIG. 2A, the displacement actuator 135 can disengage from or otherwise release the displacement member 140. This can allow the resilient bias member 125 to expand and bias the heat sink 115 toward the heating element 120 and return to the first state where again, heat can be transferred away from the heating element 120 and into the heat sink 115 to avoid inadvertent damage to nearby tissue during maneuvering of the jaw assembly 145 into position with the next piece of tissue to be effected/treated. This process of moving the heat sink 115 into and out of (e.g., more or less) thermal conduction with the heating element 120 can be repeated to maximize cutting performance while minimizing inadvertent damage to nearby tissues during maneuvering.

The resilient bias member 125 can include, among other things, a spring, a contained or carried fluid (e.g., hydraulic or pneumatic), a resilient or other deflecting member, a compressible material, or any combination thereof, that pushes the heat sink 115 toward the heating element 120. The compressible material can include at least one of a rubber, a fluid, a sponge, or silicone. Spring types can include, for example, a coil spring or leaf spring arranged between the second jaw 110 and the movable heat sink 115, or a torsion spring arranged about the pivot.

The resilient bias member 125 can itself be a second heat sink to further aid in dissipation of heat away from the heat sink 115, and therefore away from the heating element 120, when the first jaw 105 and the second jaw 110 are in the open position. The resilient bias member 125 can comprise a thermally conductive material and can at least partially thermally couple with the heat sink 115 when in the first state such as to provide an additional heat sinking function to promote efficient dissipation of a portion of the heat away from the heat sink 115 for rapid cooling of the heating element 120.

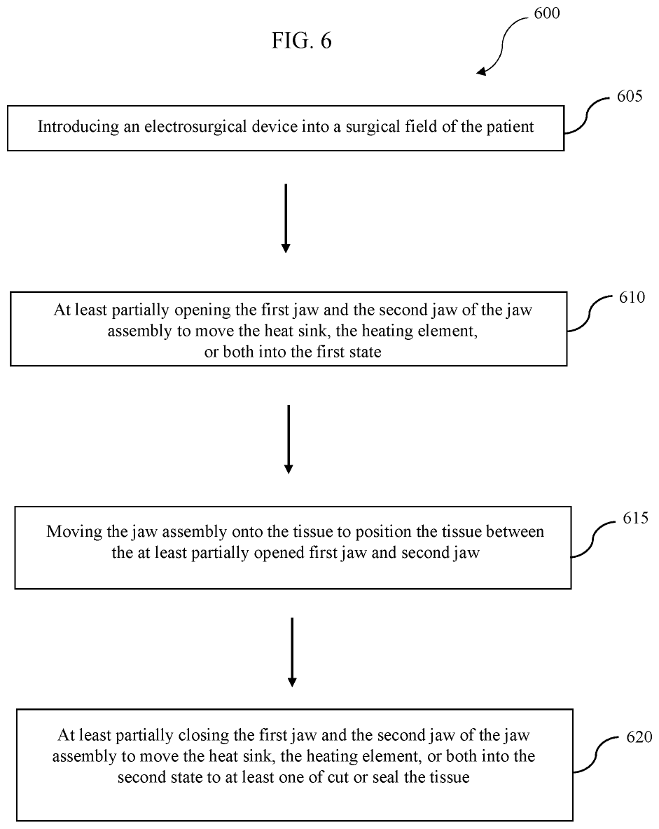

Referring to FIG. 6, a method 600 for treating a tissue of a patient comprises introducing electrosurgical device comprising a forceps into a surgical field of the patient (605). The first jaw 105 and second jaw 110 of the jaw assembly 145 can be opened to move the heat sink 115, the heating element 120, or both into the first state (610) in which the heat sink 115 can be located against or in close proximity to the heating element 120 to help dissipate heat therefrom. The jaw assembly 145 can be moved onto the tissue to position the tissue between the opened first jaw 105 and second jaw 110 (615). The first jaw 105 and the second jaw 110 of the jaw assembly 145 can be at least partially closed to move the heat sink 115, the heating element 120, or both into the second state (620) in which the heat sink 115 can be located farther from the heating element 120 to permit the heating element 120 to be heated to deliver heat to the tissue between the first jaw 105 and second jaw 110.

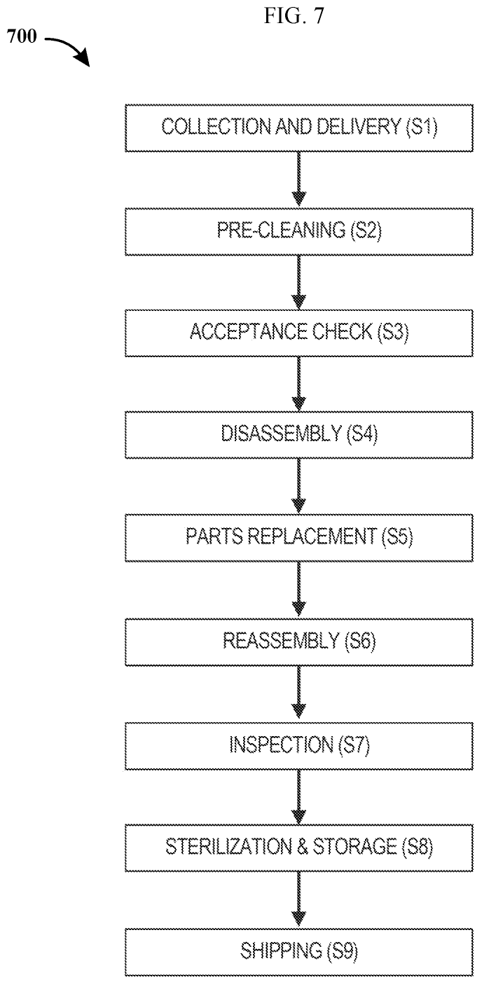

FIG. 7 is a flowchart indicating a reprocessing method 700 for any of the forceps or jaws described herein and will be described generally as the electrosurgical device 100. The electrosurgical device 100 described above may be disposed of after one use, or may be used repeatedly in multiple treatments, such as surgeries or medical procedures. In the case of a configuration that is used repeatedly, for example, the reprocessing method 700 shown in FIG. 7 can be used or may be required. The reprocessing methods described herein can be used with any of the forceps or jaws described herein, although other reprocessing methods may also be used with any of the forceps or jaws described herein.

The used electrosurgical device 100 can be collected after the electrosurgical device 100 has been used for treatment. The used electrosurgical device 100 can be delivered to a reprocessing facility (Step S1). At this time, the used electrosurgical device 100 can be transported in a dedicated container to prevent contamination of the electrosurgical device 100.

A reprocessing technician can clean and sterilize the collected and transported used electrosurgical device 100 (Step S2). Specifically, in cleaning the electrosurgical device 100, deposits adhering to the jaws of the electrosurgical device 100 are removed by using a brush. After that, to remove potentially pathogenic microorganisms derived from blood or body fluid, a cleaning solution such as an isopropanol-containing cleaning agent, proteolytic enzyme detergent, or alcohol is applied to the jaws to further clean the electrosurgical device 100. The cleaning liquid is not limited to the cleaning liquid described above, and other cleaning liquids may be used. Further, in the sterilization of the electrosurgical device 100, to sterilize the pathogenic microorganisms or other contamination adhering to the jaws, any one or more of high-pressure steam sterilization, ethylene oxide gas sterilization, gamma ray sterilization, ultraviolet radiation sterilization, hydrogen peroxide, or hydrogen peroxide low temperature sterilization can be used. The jaws are reusable and may be easy to clean.

The technician performs an acceptance check of the used electrosurgical device 100 (Step S3). The technician checks whether the used electrosurgical device 100 has significant defects or the used electrosurgical device 100 exceeds a maximum number of reprocessing. In particular, the coating to the first jaw, the second jaw, and the heat sink may be examined and the bias member, the heat sink, and the heating element may be examined and replaced as needed.

Next, the user disassembles or removes portions of the used electrosurgical device 100 to be replaced (Step S4). Specifically, if the bias member 125 loses its ability to bias the heat sink 115 toward the heating element 120, the bias member 125 can be removed and replaced during a step S5. If it is difficult to access the bias member 125 in the lower jaw 110, the jaws assembly 145 could be separated or at least partially separated from the electrosurgical device 100 to provide better access to the bias member.

After step S5, the user reassembles the electrosurgical device 100, if required. (Step S6). In some examples, Step S6 can include adding an identifier to indicate the device has been modified from its original condition, such as a adding a label or other marking to designate the device as reprocessed, refurbished or remanufactured.

After step S6, the user inspects and tests the reprocessed electrosurgical device 100 (step S7). Specifically, the user verifies that the reprocessed electrosurgical device 100 has the same effectiveness and safety as the original product by various functional tests, such as electrical testing of the performance of the heating element 120, the heat sink 115, and the bias member 125. There is an advantage that it is easy to verify the performance in Step S7.

After Step S7, the user sequentially performs a sterilization and storage (Step S8), and shipping (Step S9) of the reprocessed electrosurgical device 100. In the Step S8, a sterilization treatment using a sterilizing gas such as ethylene oxide gas or propylene oxide gas is applied to the reprocessed electrosurgical device 100 and it is stored in a storage container until use.

Steps S1 to S9 described above are executed to achieve reprocessing of the electrosurgical device 100.

Each of the non-limiting examples described herein can stand on its own or can be combined in various permutations or combinations with one or more of the other examples. This Summary/Overview is intended to provide an overview of subject matter of the present patent application. It is not intended to provide an exclusive or exhaustive explanation of the invention. The detailed description is included to provide further information about the present patent application.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The following statements are intended to describe and summarize various embodiments of the invention according to the foregoing description in the specification.

### Examples #1

Example 1 is an electrosurgical device for cutting or sealing tissue of a patient, the electrosurgical device comprising: a jaw assembly comprising: a first jaw having a first distal end and a first proximal end; a second jaw having a second distal end and a second proximal end, wherein the first proximal end is movably coupled to the second proximal end to provide an end effector forceps including the first and second jaws; a heating element configured to heat to a temperature sufficient for cutting or sealing the tissue of the patient, wherein the heating element is disposed with the second jaw; and a heat sink disposed with the second jaw adjacent to the heating element, wherein the heat sink, the heating element, or both move relative to each other between a first state that at least partially thermally couples the heat sink and the heating element to at least partially dissipate heat away from the heating element and a second state that at least partially thermally de-couples the heat sink from the heating element.

In Example 2, the subject matter of Example 1 optionally includes wherein, when the first jaw and the second jaw are in a closed position or moved toward the closed position, the first jaw actuates displacement of the heat sink into the second state and the heating element is energized to deliver heat to tissue captured between the first jaw and the second jaw to effect the cutting or sealing of the tissue.

In Example 3, the subject matter of any one or more of Examples 1-2 optionally include wherein, when the first jaw and the second jaw are in an open position or moved toward the open position, the first jaw actuates release of the heat sink from the second state and the heat sink moves into the first state.

In Example 4, the subject matter of any one or more of Examples 1-3 optionally include wherein the first jaw includes a displacement actuator that engages the heat sink when the first jaw and the second jaw are in a closed position or moving toward the closed position, wherein the displacement actuator moves the heat sink toward the second state.

In Example 5, the subject matter of any one or more of Examples 1-4 optionally include wherein the first jaw includes a displacement actuator that engages the heating element, the heat sink, or both when the first jaw and the second jaw are in a closed position or moved toward the closed position to move the heating element, the heat sink, or both toward the second state.

In Example 6, the subject matter of any one or more of Examples 4-5 optionally include wherein the heat sink comprises a displacement member, wherein the displacement actuator contacts the displacement member to cause the heat sink to pivot away from the heating element in the second state.

In Example 7, the subject matter of any one or more of Examples 5-6 optionally include wherein the heat sink comprises a displacement member, wherein the displacement actuator contacts the displacement member to cause the heat sink to pivot away from the heating element in the second state.

In Example 8, the subject matter of any one or more of Examples 1-7 optionally include wherein the heat sink is at least one of pivotably or slidably attached to the second jaw at an opposite end of the second jaw from the displacement actuator.

In Example 9, the subject matter of any one or more of Examples 1-8 optionally include wherein the second jaw further comprises a resilient bias member positioned adjacent to the heat sink, wherein the resilient bias member is configured to bias the heat sink toward the first state with the heating element when the first jaw and the second jaw are in an open position or moved toward the open position.

In Example 10, the subject matter of Example 9 optionally includes wherein the resilient bias member is at least partially thermally coupled to the heat sink to at least partially dissipate heat away from the heat sink when the first jaw and the second jaw are in the first state.

In Example 11, the subject matter of Example 10 optionally includes wherein the resilient bias member comprises a conductive material that dissipates a portion of heat away from the heat sink.

In Example 12, the subject matter of any one or more of Examples 1-11 optionally include wherein the first jaw, the second jaw, the heat sink, or a combination thereof further comprise a coating.

In Example 13, the subject matter of Example 12 optionally includes wherein the coating is a moisture wicking material applied to one or more of the first jaw, the second jaw, or the heat sink.

In Example 14, the subject matter of any one or more of Examples 12-13 optionally include wherein the coating is a non-stick material.

In Example 15, the subject matter of any one or more of Examples 12-14 optionally include wherein the coating comprises one or more of PTFE, HMDSO, ceramics, or aluminum oxide.

In Example 16, the subject matter of any one or more of Examples 12-15 optionally include wherein the coating prevents a phase change of the patient's body fluids to steam as the heat sink and the resilient bias member dissipates a portion of heat away from the heating element.

In Example 17, the subject matter of any one or more of Examples 9-16 optionally include wherein the resilient bias member comprises a compressible material.

In Example 18, the subject matter of Example 17 optionally includes wherein the compressible material is at least one of a spring, rubber, a fluid, a sponge, silicon, or a deflecting member disposed with the second jaw.

In Example 19, the subject matter of any one or more of Examples 1-18 optionally include wherein the heating element includes a first resistive electrode configured to heat to a treatment temperature that seals or cuts the tissue of the patient when the tissue is placed between the first jaw and the second jaw in the closed position.

In Example 20, the subject matter of Example 19 optionally includes wherein the first resistive electrode provides resistive heat cutting or sealing when heated to between approximately 200° C. to approximately 350° C.

In Example 21, the subject matter of Example 20 optionally includes wherein the first resistive electrode provides resistive heat cutting or sealing when heated to between approximately 260° C. to approximately 300° C.

In Example 22, the subject matter of any one or more of Examples 1-21 optionally include wherein the heating element and the heat sink contact each other in the first state when the first jaw and the second jaw are in an open position or moved toward an open position.

In Example 23, the subject matter of any one or more of Examples 1-22 optionally include wherein a thermal conductivity of the heat sink is greater than a thermal conductivity of the heating element.

In Example 24, the subject matter of any one or more of Examples 22-23 optionally include wherein placing the heat sink in the first state with respect to the heating element reduces the temperature of the heating element to approximately ambient temperature.

In Example 25, the subject matter of any one or more of Examples 22-24 optionally include wherein the temperature of the heating element in the heat sinking position is reduced from the treatment temperature to approximately ambient temperature within approximately 0.25 seconds to approximately 3 seconds.

In Example 26, the subject matter of any one or more of Examples 24-25 optionally include wherein the temperature of the heating element is reduced from the treatment temperature to between approximately 37° C. and approximately 42° C.

In Example 27, the subject matter of any one or more of Examples 1-26 optionally include wherein the heating element is configured to receive an electrical energy signal to deliver electromagnetic energy to tissue to seal or cut the tissue.

In Example 28, the subject matter of any one or more of Examples 1-27 optionally include a pivot member coupled to the heat sink to permit displacement of the heat sink away from the heating element into the standby position when the first jaw and the second jaw are in the closed position.

In Example 29, the subject matter of Example 28 optionally includes wherein the pivot member coupled to the heat sink moves the heat sink toward the heating element into the first state when the first and second jaw are in the open position or moving towards the open position.

In Example 30, the subject matter of any one or more of Examples 1-29 optionally include a displacement actuator coupled to the proximal end of the first jaw, wherein the displacement actuator prevents the patient's tissue from advancing further into the jaw assembly when the tissue is placed between the first jaw and the second jaw.

In Example 31, the subject matter of any one or more of Examples 1-30 optionally include wherein the heating element is configured to receive energy from a power source.

Example 32 is there electrosurgical device of Example 31, wherein the energy received by the heating element from the power source heats the heating element.

Example 33 is an electrosurgical device for treating a tissue, the electrosurgical device comprising: an end effector including a jaw assembly comprising a heating element configured to receive energy; and a heat sink configured to move relative to the heating element of the jaw assembly, wherein the heat sink moves between a first state that at least partially thermally couples the heat sink and the heating element to at least partially dissipate heat away from the heating element and a second state that at least partially thermally de-couples the heat sink from the heating element; and wherein the heating element cools when the heat sink is in the first state and the heating element heats when the heat sink is in the second state.

In Example 34, the subject matter of Example 33 optionally includes wherein the heating element includes a first resistive electrode.

In Example 35, the subject matter of any one or more of Examples 33-34 optionally include wherein a thermal conductivity of the heat sink is greater than a thermal conductivity of the heating element.

In Example 36, the subject matter of any one or more of Examples 33-35 optionally include a pivot member coupled to the heat sink to permit displacement of the heat sink away from the heating element into the second state.

In Example 37, the subject matter of any one or more of Examples 33-36 optionally include wherein the electrosurgical device is a surgical forceps.

Example 38 is a method of treating a tissue of a patient with an electrosurgical device, the method comprising: receiving an input at a jaw assembly of the electrosurgical device to move a heat sink relative to a heating element of the jaw assembly between a first state and a second state, wherein the heat sink and the heating element are more thermally coupled in the first state than in the second state; and receiving a treatment energy at the heating element in the second state, the treatment energy configured to treat the tissue.

In Example 39, the subject matter of Example 38 optionally includes wherein receiving the treatment energy at the heating element includes receiving radiofrequency energy.

In Example 40, the subject matter of any one or more of Examples 38-39 optionally include wherein the heating element is configured to cut or seal the tissue when receiving the treatment energy.

In Example 41, the subject matter of Example 40 optionally includes wherein receiving the treatment energy includes receiving an energy amount or energy waveform that is configured to cut the tissue with the heating element.

In Example 42, the subject matter of any one or more of Examples 40-41 optionally include wherein receiving the treatment energy includes receiving an energy amount or energy waveform that is configured to cut, seal, ablate, dessicate, fulgrate or necrose the tissue with the heating element.

### Examples #2

Example 1 is an electrosurgical device for treating tissue of a patient, the electrosurgical device comprising: an end effector including a heating element configured to receive energy to cut tissue; and a heat sink configured to selectively transfer heat away from the heating element of the end effector; wherein the heat sink and the end effector are configured to move between a first relative position where the heat sink and the heating element are in a first thermal exchange state and a second relative position where the heat sink and the heating element are in a second thermal exchange state; and wherein the heating element is configured to heat-up in the first relative position and the heat sink is configured to cool-down the heating element in the second relative position.

In Example 2, the subject matter of Example 1 optionally includes wherein the heating element comprises a first resistive electrode.

In Example 3, the subject matter of any one or more of Examples 1-2 optionally include an actuator to push the heat sink into the first relative position away from the heating element.

In Example 4, the subject matter of Example 3 optionally includes a displacement member coupled to the heat sink to permit sliding or pivoting of the heat sink when engaged with the actuator and to produce the first relative position and the second relative position.

In Example 5, the subject matter of Example 4 optionally includes a biasing member to push the heat sink into the second relative position toward the heating element.

In Example 6, the subject matter of any one or more of Examples 1-5 optionally include wherein the electrosurgical device is a surgical forceps.

In Example 7, the subject matter of Example 6 optionally includes wherein the surgical forceps comprises: a first jaw; and a second jaw pivotable relative to the first jaw; wherein the heating element comprises a wire extending from the first jaw; and wherein at least one of the first jaw and the second jaw comprises a seal electrode.

In Example 8, the subject matter of Example 7 optionally includes wherein the second jaw includes an actuator to push the heat sink away from the heating element.

In Example 9, the subject matter of Example 8 optionally includes a biasing member to push the heat sink toward the heating element.

In Example 10, the subject matter of any one or more of Examples 1-9 optionally include wherein the heating element is moveable and the heat sink is stationary relative to the electrosurgical device.

In Example 11, the subject matter of any one or more of Examples 1-10 optionally include wherein a thermal conductivity of the heat sink is greater than a thermal conductivity of the heating element.

In Example 12, the subject matter of any one or more of Examples 1-11 optionally include wherein the first thermal exchange state is less thermally conductive than the second thermal exchange state.

In Example 13, the subject matter of Example 12 optionally includes wherein: in the first relative position the heating element and the heat sink are not in physical contact with each other; and in the second relative position the heating element and the heat sink are in physical contact with each other.

In Example 14, the subject matter of any one or more of Examples 12-13 optionally include wherein: in the first relative position the heating element and the heat sink are at least partially thermally un-coupled; and in the second relative position the heating element and the heat sink are at least partially thermally coupled.

In Example 15, the subject matter of Example 14 optionally includes wherein: in the first relative position the heating element and the heat sink are in physical contact over a first surface area; and in the second relative position the heating element and the heat sink are in physical contact over a second surface area; wherein the first surface area is less than the second surface area.

Example 16 is a method of treating a tissue of a patient with an electrosurgical device, the method comprising: receiving a treatment energy at a heating element of a jaw assembly of the electrosurgical device to heat the heating element to a hotter state; treating the tissue with the heating element; moving a heat sink and the heating element of the jaw assembly from a first relative position to a second relative position, wherein the heat sink and the heating element are more thermally coupled in the second relative position than in the first relative position; and transferring heat from the heating element to the heat sink to cool the heating element to a cooler state.

In Example 17, the subject matter of Example 16 optionally includes wherein receiving the treatment energy at the heating element includes receiving radiofrequency energy.

In Example 18, the subject matter of any one or more of Examples 16-17 optionally include wherein receiving the treatment energy includes receiving an energy amount or energy waveform that is configured to cut the tissue with the heating element.

In Example 19, the subject matter of any one or more of Examples 16-18 optionally include wherein receiving the treatment energy includes receiving an energy amount or energy waveform that is configured to cut, seal, ablate, dessicate, fulgrate or necrose the tissue with the heating element.

In Example 20, the subject matter of any one or more of Examples 16-19 optionally include receiving an input to move the heat sink and the heating element from the second relative position to the first relative position before receiving the treatment energy.

In Example 21, the subject matter of Example 20 optionally includes wherein receiving the input comprises rotating a jaw of the jaw assembly to push an actuator that displaces the heat sink.

In Example 22, the subject matter of Example 21 optionally includes actuating a first jaw of the jaw assembly to push the heat sink away from the heating element.

In Example 23, the subject matter of any one or more of Examples 21-22 optionally include wherein rotating a jaw of the jaw assembly to push the actuator that displaces the heat sink comprises pushing a pivot member on the heat sink with the actuator.

In Example 24, the subject matter of any one or more of Examples 20-23 optionally include releasing the input; and activating a biasing force to push the heat sink into engagement with the heating element.

In Example 25, the subject matter of Example 24 optionally includes wherein activating the biasing force to push the heat sink into engagement with the heating element comprises uncompressing a resilient body.

In Example 26, the subject matter of any one or more of Examples 20-25 optionally include wherein receiving the input to move the heat sink and the heating element from the second relative position to the first relative position before receiving the treatment energy comprises sliding the heat sink.

In Example 27, the subject matter of any one or more of Examples 20-26 optionally include wherein receiving the input comprises moving the heating element toward the heat sink.

In Example 28, the subject matter of any one or more of Examples 16-27 optionally include wherein in the second relative position the heating element and the heat sink are in contact and in the first relative position the heating element and the heat sink are not in contact.

In Example 29, the subject matter of any one or more of Examples 16-28 optionally include wherein in the second relative position the heating element and the heat sink are in contact over a greater surface area than in the first relative position.

In Example 30, the subject matter of any one or more of Examples 16-29 optionally include activating a seal electrode on a jaw of the jaw assembly to treat the tissue.

Example 31 is an electrosurgical device for cutting or sealing tissue of a patient, the electrosurgical device comprising: a jaw assembly comprising: a first jaw having a first distal end and a first proximal end; and a second jaw having a second distal end and a second proximal end, wherein the first proximal end is movably coupled to the second proximal end to provide an end effector forceps including the first jaw and the second jaw; a heating element configured to heat to a temperature sufficient for cutting or sealing the tissue of the patient, wherein the heating element is connected to the second jaw; and a heat sink disposed connected to the second jaw adjacent to the heating element; wherein the heat sink, the heating element, or both move relative to each other between a first state that at least partially thermally couples the heat sink and the heating element to at least partially dissipate heat away from the heating element and a second state that at least partially thermally de-couples the heat sink from the heating element to allow heat to accumulate within the heating element when activated.

In Example 32, the subject matter of Example 31 optionally includes wherein: when the first jaw and the second jaw are in a closed position or moved toward the closed position, the first jaw actuates displacement of the heat sink into the second state and the heating element can be energized to deliver heat to tissue captured between the first jaw and the second jaw to effect cutting or sealing of the tissue; and when the first jaw and the second jaw are in an open position or moved toward the open position, the first jaw actuates release of the heat sink from the second state and the heat sink moves into the first state.

In Example 33, the subject matter of any one or more of Examples 31-32 optionally include wherein the first jaw includes a displacement actuator that engages the heating element, the heat sink, or both when the first jaw and the second jaw are in a closed position or moved toward the closed position to move the heating element, the heat sink, or both toward the second state.

In Example 34, the subject matter of Example 33 optionally includes wherein the heat sink comprises a displacement member, wherein the displacement actuator contacts the displacement member to cause the heat sink to pivot away from the heating element in the second state.

In Example 35, the subject matter of any one or more of Examples 33-34 optionally include wherein the heat sink is at least one of pivotably or slidably attached to the second jaw to engage with the displacement actuator.

In Example 36, the subject matter of any one or more of Examples 31-35 optionally include wherein the second jaw further comprises a resilient bias member positioned adjacent to the heat sink, wherein the resilient bias member is configured to bias the heat sink toward the first state with the heating element when the first jaw and the second jaw are in an open position or moved toward the open position.

In Example 37, the subject matter of Example 36 optionally includes wherein the resilient bias member is at least partially thermally coupled to the heat sink to at least partially dissipate heat away from the heat sink when the first jaw and the second jaw are in the first state, wherein the resilient bias member comprises a conductive material that dissipates a portion of heat away from the heat sink.

In Example 38, the subject matter of any one or more of Examples 36-37 optionally include wherein the resilient bias member comprises a compressible material that is at least one of a spring, rubber, a fluid, a sponge, silicon, or a deflecting member disposed with the second jaw.

In Example 39, the subject matter of any one or more of Examples 31-38 optionally include wherein the first jaw, the second jaw, the heat sink, or a combination thereof further comprise a moisture wicking material applied to one or more of the first jaw, the second jaw, or the heat sink.

In Example 40, the subject matter of any one or more of Examples 31-39 optionally include wherein the first jaw, the second jaw, the heat sink, or a combination thereof further comprise a non-stick coating comprising one or more of PTFE, HMDSO, ceramics, or aluminum oxide.

In Example 41, the subject matter of any one or more of Examples 31-40 optionally include wherein the first jaw, the second jaw, the heat sink, or a combination thereof further comprise a coating configured to prevent a phase change of body fluids of the patient to steam as the heat sink dissipates a portion of heat away from the heating element.

In Example 42, the subject matter of any one or more of Examples 32-41 optionally include wherein the heating element includes a first resistive electrode configured to heat to a treatment temperature that seals or cuts the tissue of the patient when the tissue is placed between the first jaw and the second jaw in the closed position.

In Example 43, the subject matter of Example 42 optionally includes ° C.

In Example 44, the subject matter of any one or more of Examples 42-43 optionally include wherein the heating element and the heat sink contact each other in the first state when the first jaw and the second jaw are in an open position or moved toward an open position.

In Example 45, the subject matter of any one or more of Examples 42-44 optionally include wherein a thermal conductivity of the heat sink is greater than a thermal conductivity of the heating element.

In Example 46, the subject matter of any one or more of Examples 44-45 optionally include ° C.

In Example 47, the subject matter of Example 46 optionally includes seconds.

In Example 48, the subject matter of any one or more of Examples 31-47 optionally include wherein the heating element is configured to receive an electrical energy signal from a power source to deliver electromagnetic energy to tissue to seal or cut the tissue.

In Example 49, the subject matter of any one or more of Examples 32-48 optionally include a pivot member coupled to the heat sink to permit displacement of the heat sink away from the heating element when the first jaw and the second jaw are in the closed position, wherein the pivot member coupled to the heat sink moves the heat sink toward the heating element into the first state when the first jaw and the second jaw are in the open position or moving towards the open position.

In Example 50, the subject matter of any one or more of Examples 31-49 optionally include a displacement actuator coupled to a proximal end of the first jaw, wherein the displacement actuator prevents tissue of the patient from advancing further into the jaw assembly when the tissue is placed between the first jaw and the second jaw.

The claimed invention is:

1. An electrosurgical device for treating tissue of a patient, the electrosurgical device comprising:
an end effector including a heating element configured to receive energy to cut tissue; and
a heat sink configured to selectively transfer heat away from the heating element of the end effector;
wherein the heat sink and the end effector are configured to move between a first relative position where the heat sink and the heating element are in a first thermal exchange state and a second relative position where the heat sink and the heating element are in a second thermal exchange state; and
wherein the heating element is configured to heat-up in the first relative position and the heat sink is configured to cool-down the heating element in the second relative position.

2. The electrosurgical device of claim 1, wherein the heating element comprises a first resistive electrode.

3. The electrosurgical device of claim 1, further comprising an actuator to push the heat sink into the first relative position away from the heating element.

4. The electrosurgical device of claim 3, further comprising a displacement member coupled to the heat sink to permit sliding or pivoting of the heat sink when engaged with the actuator and to produce the first relative position and the second relative position.

5. The electrosurgical device of claim 4, further comprising a biasing member to push the heat sink into the second relative position toward the heating element.

6. The electrosurgical device of claim 1, wherein the electrosurgical device is a surgical forceps.

7. The electrosurgical device of claim 6, wherein the surgical forceps comprises:
a first jaw; and
a second jaw pivotable relative to the first jaw;
wherein the heating element comprises a wire extending from the first jaw; and
wherein at least one of the first jaw and the second jaw comprises a seal electrode.

8. The electrosurgical device of claim 7, wherein the second jaw includes an actuator to push the heat sink away from the heating element.

9. The electrosurgical device of claim 8, further comprising a biasing member to push the heat sink toward the heating element.

10. The electrosurgical device of claim 1, wherein the heating element is moveable and the heat sink is stationary relative to the electrosurgical device.

11. The electrosurgical device of claim 1, wherein a thermal conductivity of the heat sink is greater than a thermal conductivity of the heating element.

12. The electrosurgical device of claim 1, wherein the first thermal exchange state is less thermally conductive than the second thermal exchange state.

13. The electrosurgical device of claim 12, wherein:
in the first relative position the heating element and the heat sink are not in physical contact with each other; and
in the second relative position the heating element and the heat sink are in physical contact with each other.

14. The electrosurgical device of claim 12, wherein:
in the first relative position the heating element and the heat sink are at least partially thermally un-coupled; and
in the second relative position the heating element and the heat sink are at least partially thermally coupled.

15. The electrosurgical device of claim 14, wherein:
in the first relative position the heating element and the heat sink are in physical contact over a first surface area; and
in the second relative position the heating element and the heat sink are in physical contact over a second surface area;
wherein the first surface area is less than the second surface area.

16. A method of treating a tissue of a patient with an electrosurgical device, the method comprising:
receiving a treatment energy at a heating element of a jaw assembly of the electrosurgical device to heat the heating element to a hotter state;
treating the tissue with the heating element;
moving a heat sink and the heating element of the jaw assembly from a first relative position to a second relative position, wherein the heat sink and the heating element are more thermally coupled in the second relative position than in the first relative position; and
transferring heat from the heating element to the heat sink to cool the heating element to a cooler state.

17. The method of claim 16, wherein receiving the treatment energy at the heating element includes receiving radiofrequency energy.

18. The method of claim 16, wherein receiving the treatment energy includes receiving an energy amount or energy waveform that is configured to cut the tissue with the heating element.

19. The method of claim 16, wherein receiving the treatment energy includes receiving an energy amount or energy waveform that is configured to cut, seal, ablate, dessicate, fulgrate or necrose the tissue with the heating element.

20. The method of claim 16, further comprising:
receiving an input to move the heat sink and the heating element from the second relative position to the first relative position before receiving the treatment energy.

21. The method of claim 20, wherein receiving the input comprises rotating a jaw of the jaw assembly to push an actuator that displaces the heat sink.

22. The method of claim 21, further comprising actuating a first jaw of the jaw assembly to push the heat sink away from the heating element.

23. The method of claim 21, wherein rotating a jaw of the jaw assembly to push the actuator that displaces the heat sink comprises pushing a pivot member on the heat sink with the actuator.

24. The method of claim 20, further comprising:
releasing the input; and
activating a biasing force to push the heat sink into engagement with the heating element.

25. The method of claim 24, wherein activating the biasing force to push the heat sink into engagement with the heating element comprises uncompressing a resilient body.

26. The method of claim 20, wherein receiving the input to move the heat sink and the heating element from the second relative position to the first relative position before receiving the treatment energy comprises sliding the heat sink.

27. The method of claim 20, wherein receiving the input comprises moving the heating element toward the heat sink.

28. The method of claim 16, wherein in the second relative position the heating element and the heat sink are in contact and in the first relative position the heating element and the heat sink are not in contact.

29. The method of claim 16, wherein in the second relative position the heating element and the heat sink are in contact over a greater surface area than in the first relative position.

30. The method of claim 16, further comprising activating a seal electrode on a jaw of the jaw assembly to treat the tissue.

31. An electrosurgical device for cutting or sealing tissue of a patient, the electrosurgical device comprising:
a jaw assembly comprising:
a first jaw having a first distal end and a first proximal end; and
a second jaw having a second distal end and a second proximal end, wherein the first proximal end is movably coupled to the second proximal end to provide an end effector forceps including the first jaw and the second jaw;
a heating element configured to heat to a temperature sufficient for cutting or sealing the tissue of the patient, wherein the heating element is connected to the second jaw; and
a heat sink disposed connected to the second jaw adjacent to the heating element;
wherein the heat sink, the heating element, or both move relative to each other between a first state that at least partially thermally couples the heat sink and the heating element to at least partially dissipate heat away from the heating element and a second state that at least partially thermally de-couples the heat sink from the heating element to allow heat to accumulate within the heating element when activated.
